(19) United States
(12) Patent Application Publication
(10) Pub. No.: US 2010/0075377 A1
(43) Pub. Date: Mar. 25, 2010

(54) TETRAMERIZING POLYPEPTIDES AND METHODS OF USE

(76) Inventors: JAMES W. WEST, SANTA BARBARA, CA (US); CAMERON S. BRANDT, SEATTLE, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(21) Appl. No.: 12/553,232

(22) Filed: Sep. 3, 2009

Related U.S. Application Data
(63) Continuation-in-part of application No. 11/734,886, filed on Apr. 13, 2007.
Application No. 11/734,886 is a continuation-in-part of application No. 11/735,328, filed on Apr. 13, 2007, now abandoned.
(60) Provisional application No. 60/791,626, filed on Apr. 13, 2006.
(60) Provisional application No. 60/791,627, filed on Apr. 13, 2006.

(51) Int. Cl. C12P 21/02; C07K 14/00

(52) U.S. Cl. 435/69.1; 530/350

(57) ABSTRACT

The present invention relates to a method of preparing a tetrameric protein comprising culturing a host cell transformed or transfected with an expression vector encoding a fusion protein comprising a vasodialator-stimulated phosphoprotein (VASP) domain and a heterologous protein. In one embodiment, the heterologous protein is a membrane protein, the portion of the heterologous protein that included in the fusion protein is the extracellular domain of that protein, and the resulting fusion protein is soluble. The method can be used to produced homo- and hetero-tetrameric proteins. The present invention also encompasses DNA molecules, expression vectors, and host cells used in the present method and fusion proteins produced by the present method.

### RELATED APPLICATIONS

This application is a continuation-in-part of U.S. application Ser. No. 11/734,886, filed Apr. 13, 2007, which claims the benefit of U.S. Provisional Application Ser. No. 60/791,626, filed Apr. 13, 2006, each incorporated herein reference. This application is also a continuation-in-part of U.S. application Ser. No. 11/735,328, filed Apr. 13, 2007, which claims the benefit of U.S. Provisional Application Ser. No. 60/791,627, filed Apr. 13, 2006, each incorporated herein by reference.

### BACKGROUND OF THE INVENTION

The present invention relates to polypeptides able to form multimers, particularly tetramers, and the manufacture and use of such polypeptides.

A. Coiled-Coils

A basic component of the quaternary structure of the present multimerizing polypeptides is the coiled-coil (reviewed in Müller et al., (2000) Meth. Enzymol. 328: 261-283). Coiled-coils are protein domains that take the shape of gently twisted, ropelike bundles. The bundles contain two to five α helices in parallel or antiparallel orientation. The essential feature of many coiled-coil sequences is a seven-residue, or heptad, repeat (commonly labeled (abcdefg)ₙ) with the first (a) and fourth (d) positions usually occupied by hydrophobic amino acids. The remaining amino acids of the coiled-coil structure are generally polar, where proline is usually excluded due to its disruptive effect on helical architecture.

This characteristic heptad repeat (also known as a 3,4 hydrophobic repeat) is what forms the structure of the coiled-coil domain, with each residue sweeping about 100°. This results in the seven residues of the heptad repeat falling short of two full turns by about 27°. The lag forms a gentle, left-handed hydrophobic stripe of residues running down the α helix and the coiled-coil structure forms when these hydrophobic stripes associate. Deviations from the regular 3,4 spacing of nonpolar residues changes the angle of the hydrophobic stripe with respect to the α helix axis, altering the crossing angle of the helices and destabilizing the quaternary structure. In other words, supercoiling (either left or right) results when helixes containing hydrophobic patches that occur at less than or greater than full turns associate with each other. With heptad repeats, the hydrophobic patches are just short of two full turns and result in left-handed supercoiling upon association.

Although heptad repeats are by far the most common length of repeat structure found and studied in coiled-coil sequences, other repeats lengths are also possible. Specifically, 11 residue repeats have been found in the tetrabrachion protein from the micro-organism Staphylohthermus marinus (Peters et al. (1996) J. Mol. Biol. 257: 1031). This protein has a parallel four-stranded coiled-coil with slight right-handed supercoiling. A still larger repeat has been observed in a domain of the vasodilator-stimulated phosphoprotein (VASP) which includes 15 residue repeats within the region of the protein responsible for forming tetramers. (Kühnel et al. (2004) Proc. Natl. Acad. Sci. 101: 17027). In contrast to the common heptad repeat coiled-coil structures, the supercoiling for the 15-residue repeat is right handed, rather than left handed, but it is of a similar degree.

Coiled-coil domain sequences have been fused to other heterologous protein sequences to achieve diverse experimental goals. One common use is the replacement of natural oligomerization domains with a heterologous sequence to alter oligomerization state, stability, and/or avidity. Low affinity monomers that do not naturally associate can be oligomerized in order to bind effectly to other multimeric targets. Additionally, the oligmerization domain fusion can be used to mimic the activated state of the native protein that is difficult to achieve with recombinant protein production (see, e.g., Pullen et al. (1999) Biochem. 94:6032). This approach has been particularly effective when producing only specific domains, such as the extracellular (cytoplasmic) or intracellular portion of a protein of interest. Commonly, coiled-coils are genetically fused to the protein of interested via a flexible linker that will provide access for the fusion to a large three-dimensional space. Direct fusions are used for experimental goals that require more rigid molecules, such as those used for crystallization.

A number of model coiled-coil systems have been developed based on the structural information of large structural proteins, such as myosin and tropomyosin (TM43, Lau et al. J Biol Chem; 259: 13253-13261), a group of proteins known as collectins (Hoppe et al. (1994) Protein Sci; 3:1143-1158), or of the dimerization region of DNA regulatory proteins, such as the yeast transcriptional activator protein GCN4-pl (Landschulz et al. (1988) Science; 240:1759-1764). This last structure is often referred to as a “leucine zipper” or LZ. Derivative model systems from the TM43 have been made, specifically where one leucine per heptad has been switched to phenylalanine. This structure is known as a “phenylalanine zipper” or FZ (Thomas et al. Prog Colloid Polymer Sci; 99: 24-30). A third type of well-known derivative of the LZ is the isoleucine zipper (IZ) (Harbury et al. (1994) Nature 371:80-83).

An important constraint of model coiled-coils is the ability to be produced in the expression host. The lack of disulfide bonds in coiled-coil structures aids their production in heterologous expression systems. However, de novo designed sequences tend to be sensitive to proteolysis. Even if effectively expressed, the relative lack of effectiveness as compared to natural sequences reflects the gaps in the current knowledge about all variables involved in protein interaction (Arndt et al. (2002) Structure 10: 1235-1248). Additionally, the use of model sequences is problematic when the goal of the fusion protein produced is a biologically functional protein.

B. Vasodialator-Stimulated Phosphoprotein (VASP)

As mentioned above, this protein has been shown through crystallization to include a tetramerization region comprising 15 residue (quindecad) repeats that result in a parallel right-handed coiled-coil structure that has a similar degree of supercoiling as the left handed coiled coils that result from heptad repeats (see FIG. 2). This structure is further stabilized with salt bridges, particularly strong hydrogen bonds that form between two charged amino acid residues.

In more detail, two consecutive 15 repeats are seen within the protein, where seven (positions a, b, d, e, f, j, and o) are identical between the two repeats and four (positions c, h, i, and l) are conservative changes that preserve either the charge and/or the hydrophobicity of the substituted amino acid residue. The 15-residue repeat has a pronounced pattern of repeated hydrophobic residues in positions a, d, h, and l. These residues plus the aliphatic portion of the lysine in the e position make up the hydrophobic core of the VASP tetramerizing domain. For a 15 residue repeat, the α helical phase increment overshoots four full turns by about 44° which means when the hydrophobic regions of this protein associate, it results in a right-handed superhelix not dissimilar in degree to the left-handed superhelix of heptad repeat containing α helixes. A comparison between the VASP structure and a common leucine zipper (GCN4-pLI) is shown in FIG. 2.

Another way to express the structure of this domain is that it is one heptad repeat with two four residue stutters. One or more stutters (a term of art for an insertion) are found in many coiled-coils comprising heptads and can cause an “unwinding” of the left-handed coiled-coil or even a local area of right-handed twist (see, e.g. Brown et al. (1996) Proteins 26:134). So the VASP tetramerizing domain can be described as a heptad repeat with regularly repeated four amino acid stutters that flank it. The stutters result in right handed supercoiling. Thus, if a heptad is called a 3, 4 hydrophobic repeat, the VASP domain can be called a 4, 3, 4, 4 hydrophobic repeat, the middle 3, 4 representing the heptad portion.

There remains a need in the art to adapt natural tetramerization sequences for use in the production of biologically active, recombinant fusion proteins. Accordingly, the present application describes polynucleotides and polypeptides useful for tetramerization in the recombinant protein art.

### SUMMARY OF THE INVENTION

The present invention relates to a method of preparing a multimeric protein, preferably a tetrameric protein, comprising culturing a host cell transformed or transfected with an expression vector encoding a fusion protein comprising a vasodialator-stimulated phosphoprotein (VASP) domain and a heterologous protein. In one embodiment, the heterologous protein is a membrane protein, the portion of the heterologous protein that is included in the fusion protein is the extracellular domain of that protein, and the resulting fusion protein is soluble. One such embodiment is made with the extracellular domain of the transmembrane co-stimulatory molecule, B7H1 (also known as programmed cell death 1 ligand 1 or PCD1L1). Another such molecule, zB7R1 (SEQ ID NO:18), can also be used. In a further embodiment, the fusion protein comprises a linker sequence. In still another embodiment of the present invention, the VASP domain can be used to identify sequences having similar protein structure patterns and those similar domains are used to make a fusion protein that multimerizes a heterologous protein or protein domain.

A further embodiment of the present invention is a method of preparing a soluble, homo- or hetero-tetrameric protein by culturing a host cell transformed or transfected with at least one, but up to four different expression vectors encoding a fusion protein comprising a VASP domain and a heterologous protein or protein domain. In this embodiment, the four VASP domains preferentially form a homo- or hetero-tetramer. This culturing can occur in the same or different host cells. The VASP domains can be the same or different and the fusion protein can further comprise a linker sequence. In one particular embodiment, the protein used to form the homo-tetrameric protein is the extracellular domain of B7H1 (PCD1L1). In another embodiment, the extracellular domain of zB7R1 is used (SEQ ID NO:19). The present invention also encompasses DNA sequences, expression vectors, and transformed host cells utilized in the present method and fusion proteins produced by the present method.

These and other aspects of the invention will become apparent to those persons skilled the art upon reading the details of the invention as more fully described below.

All references cited herein are incorporated by reference in their entirety.

### BRIEF DESCRIPTION OF THE DRAWINGS

FIG. 1. is a graphic representation of the structure of coiled-coil proteins and the interaction between residues within the coil and the residues between coils.

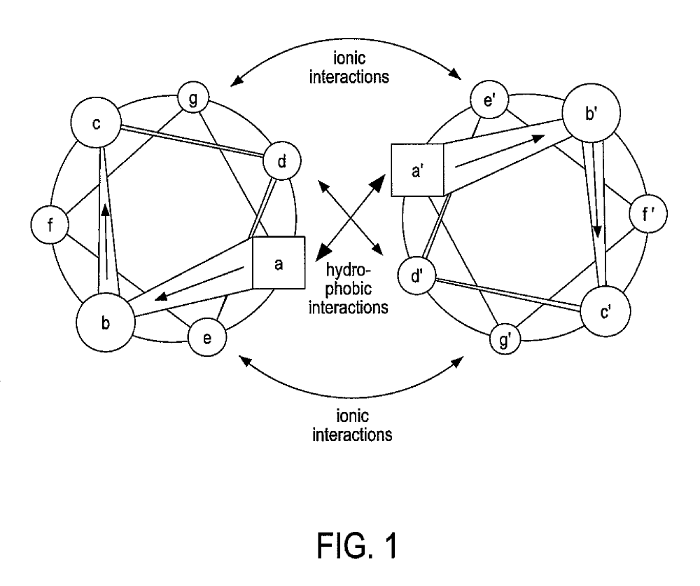

FIG. 2. is a pictoral representation of the supercoiling present in a leucine zipper and in the VASP tetramerizing domain (derived from Kühnel et al, supra).

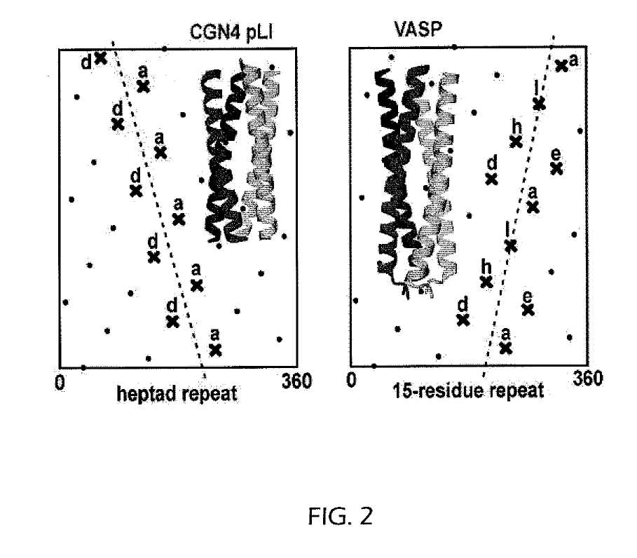

FIG. 3. is a graph documenting the binding of the B7H1-VASP fusion protein to cells expressing PD-1.

FIG. 4. shows the competition of the binding of the B7H1-VASP fusion protein and PD-1 expressing cells with other PD-1 ligands, but not with non-PD-1 binding B7 family members.

FIG. 5. illustrates the competition of the binding of labeled B7H1-VASP fusion protein to PD-1 expressing cells with unlabeled B7H1-Ig.

### DETAILED DESCRIPTION OF THE INVENTION

The present invention provides a method of preparing a multimeric, preferably tetrameric, protein by culturing a host cell transformed or transfected with an expression vector encoding a fusion protein comprising a vasodialator-stimulated phosphoprotein (VASP) domain and a heterologous protein. The invention is based on the finding that tetramerization sequences derived from certain proteins result in highly bioactive fusion proteins. This observation allowed the development of a fusion protein production method that can be utilized to produce homo- or hetero-tetrameric proteins that retain their biological activity.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a polymorphism includes a plurality of such polymorphisms, reference to “a nucleic acid molecule” includes a plurality of such nucleic acid molecules, and reference to “the method” includes reference to one or more methods, method steps, and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

In the present patent application, the term “fusion protein” is used herein to describe a protein whose sequences derive from at least two different gene sources. The sequences are genetically engineered to be transcribed and translated into one protein that comprises sequences from at least two different genes. For the present invention, one gene source is a 15 residue repeat sequence (known as the vasodialator-stimulated phosphoprotein or VASP domain) and the additional gene source or sources are one or more heterologous genes. The fusion protein can also comprise a linker sequence which will generally be located between the VASP domain and the heterologous protein sequence.

The term “heterologous” is used to describe a polynucleotide or protein that is not naturally encoded or expressed with the 15 residue repeat sequence of the VASP domain. The VASP domain can be derived from the human sequence or be an equivalent sequence from another species, and any gene source outside of this protein is considered heterologous. A heterologous protein can be a full length protein or a particular domain of a protein. The heterologous proteins of the present invention encompass both membrane bound proteins and soluble proteins and domains thereof.

The terms “polynucleotide” and “nucleic acid molecule” are used interchangeably herein to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes single-, double-stranded and triple helical molecules. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as oligomers or oligos and may be isolated from genes, or chemically synthesized by methods known in the art.

The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules, such as methylated nucleic acid molecules and nucleic acid molecule analogs. Analogs of purines and pyrimidines are known in the art. Nucleic acids may be naturally occurring, e.g. DNA or RNA, or may be synthetic analogs, as known in the art. Such analogs may be preferred for use as probes because of superior stability under assay conditions. Modifications in the native structure, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire ribose phosphodiester backbone with a peptide linkage.

Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without comprising affinity.

Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

The terms “polypeptide” and “protein”, used interchangebly herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

A “substantially isolated” or “isolated” polynucleotide is one that is substantially free of the sequences with which it is associated in nature. By substantially free is meant at least 50%, preferably at least 70%, more preferably at least 80%, and even more preferably at least 90% free of the materials with which it is associated in nature. As used herein, an “isolated” polynucleotide also refers to recombinant polynucleotides, which, by virtue of origin or manipulation: (1) are not associated with all or a portion of a polynucleotide with which it is associated in nature, (2) are linked to a polynucleotide other than that to which it is linked in nature, or (3) does not occur in nature.

Hybridization reactions can be performed under conditions of different “stringency”. Conditions that increase stringency of a hybridization reaction of widely known and published in the art. See, for example, Sambrook et al. (1989). Examples of relevant conditions include (in order of increasing stringency): incubation temperatures of 25° C., 37° C., 50° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or deionized water. Examples of stringent conditions are hybridization and washing at 50° C. or higher and in 0.1×SSC (9 mM NaCl/0.9 mM sodium citrate).

“Tₘ” is the temperature in degrees Celsius at which 50% of a polynucleotide duplex made of complementary strands hydrogen bonded in anti-parallel direction by Watson-Crick base pairing dissociates into single strands under conditions of the experiment. Tₘ may be predicted according to a standard formula, such as:

where [X⁺] is the cation concentration (usually sodium ion, Na⁺) in mol/L; (% G/C) is the number of G and C residues as a percentage of total residues in the duplex; (% F) is the percent formamide in solution (wt/vol); and L is the number of nucleotides in each strand of the duplex.

Stringent conditions for both DNA/DNA and DNA/RNA hybridization are as described by Sambrook et al. Molecular Cloning, A Laboratory Manual, 2nd Ed., Cold Spring Harbor Laboratory Press, Cold Spring Harbor, N.Y., 1989, herein incorporated by reference. For example, see page 7.52 of Sambrook et al.

The term “host cell” includes an individual cell or cell culture which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells tranfected or infected in vivo or in vitro with a recombinant vector or a polynucleotide of the invention. A host cell which comprises a recombinant vector of the invention is a “recombinant host cell”.

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger peptide is commonly cleaved to remove the secretory peptide during transit through the secretory pathway.

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

The terms “amino-terminal” (N-terminal) and “carboxyl-terminal” (C-terminal) are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” and “patient,” used interchangeably herein, refer to a mammal, including, but not limited to, murines, simians, humans, mammalian farm animals, mammalian sport animals, and mammalian pets.

### The Vasodialator-Stimulated Phosphoprotein (VASP) Domain

The present invention is a method of producing a multimeric, preferably tetrameric, protein that comprises a fusion protein comprising a VASP domain and a herterologous protein domain. VASP domains are derived from the VASP gene present in many species. Sequences are selected for their anticipated ability to form coiled-coil protein structure, as this structure is important for the ability to form multimeric protein forms. Particularly desired for the present invention is the ability of coiled-coil proteins to produce tetrameric protein structures. A particularly preferred embodiment utilizes amino acids 343 to 376 of the human VASP sequence (amino acids 5 to 38 of SEQ ID NO:2). The full length DNA sequence of this protein is SEQ ID NO: 16 and the full length polypeptide sequence of this protein is SEQ ID NO:17.

Work with other types of multimerizing sequences, for examples, the leucine zipper, has shown that a limited number of conservative amino acid substitutions (even at the d residue) can be often be tolerated in zipper sequences without the loss of the ability of the molecules to multimerize (Landschultz et al., (1989), supra). Thus, conservative changes from the native sequence for the VASP domain are contemplated within the scope of the invention. Table 1 shows the conservative changes that are anticipated to tolerated by the coiled-coil structure.

TABLE 1
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| | methionine |
| Aromatic: | phenylalanine |
| | tryptophan |
| | tyrosine |
| Small: | glycine |
| | alanine |
| | serine |
| | threonine |
| | methionine |

If more than one fusion protein is being used to produce hetero-multimeric proteins, for example, heterotetramers, the VASP domain that is used can be the same domain for both fusion proteins or different VASP domains, as long as the domains have the ability to associate with each other and form multimeric proteins.

The VASP domain can be put at either the N or C terminus of the heterologous protein of interest, based on considerations of function (i.e., whether the heterologous protein is a type I or type II membrane protein) and ease of construction of the construct. Additionally, the VASP domain can be located in the middle of the protein, effectively creating a double fusion protein with one heterologous sequence, a VASP domain, and a second heterologous sequence. The two heterologous sequences for the double fusion protein can be the same or different.

### Heterologous Proteins—Proteins of Interest

A heterologous protein of interest is selected primarily based on a desire to produce a multimeric, particularly tetrameric, version of the protein. Additionally, by utilizing only a soluble domain of the heterologous protein, a transmembrane protein can be produced in soluble form. Of particular interest with the present invention is the production of biologically active proteins of interest. One family of proteins that commonly utilizes multimers, such as tetramers, for activity is the B7 family, reviewed in Carino et al., Annu. Rev. Immunol. (2002) 20: 29 and, more recently, in Greenwald et al., Annu. Rev. Immunol. (2005) 23: 515. The genes involved in these families have key roles in the immune system, regulating T cell activation and tolerance. The genetic relationships in this family are complicated in that both positive (activating) and downregulation (deactivating) signals are present.

A key member of this family is the protein B7H1 (also known as PCD1L1 or PD-L1) which is expressed on B-cells, macrophages, dendritic cells, and T-cells. It is also expressed outside the lymphoid cells in endothelial tissues and on many kinds of tumor cells. This protein, and its interaction with it cross-receptor PD-1 has been implicated in several disease states including autoimmune disease, asthma, infectious disease, transplantation, and tumor immunity. It is a type I membrane protein with 290 amino acids and its sequence is reported in Dong et al. (1999) Nature Med. 5: 1365. The structure includes an 18 amino acid signal sequence, a 221 amino acid extracellular domain, a 21 amino acid transmembrane region, and a 31 amino acid cytoplasmic region. The full length DNA sequence of this protein is SEQ ID NO: 13 and the full length polypeptide sequence is SEQ ID NO:14. The ability to produce large quantities of these proteins while maintaining their function is a rate-limiting step in the full understanding the precise function of this family of proteins in normal and diseased tissues.

### Linker Sequences, Affinity Tag Sequences, and Signal Peptides

A protein of interest may be linked directly to another protein to form a fusion protein; alternatively, the proteins maybe separated by a distance sufficient to ensure the proteins form proper secondary and tertiary structure needed for biological activity. Suitable linker sequences will adopt a flexible extended confirmation and will not exhibit a propensity for developing an ordered secondary structure which could interact with the function domains of the fusions proteins, and will have minimal hydrophobic or charged character which could also interfere with the function of fusion domains. Linker sequences should be constructed with the 15 residue repeat in mind, as it may not be in the best interest of producing a biologically active protein to tightly constrict the N or C terminus of the heterologous sequence. Beyond these considerations, the length of the linker sequence may vary without significantly affecting the biological activity of the fusion protein. Linker sequences can be used between any and all components of the fusion protein (or expression construct) including affinity tags and signal peptides. An example linker is the GSGG sequence (SEQ ID NO: 11).

A further component of the fusion protein can be an affinity tag. Such tags do not alter the biological activity of fusion proteins, are highly antigenic, and provides an epitope that can be reversibly bound by a specific binding molecule, such as a monoclonal antibody, enabling repaid detection and purification of an expressed fusion protein. Affinity tages can also convey resistance to intracellular degradation if proteins are produced in bacteria, like E. coli. An exemplary affinity tag is the FLAG Tag (SEQ ID NO: 15) or the HIS₆ Tag (SEQ ID NO: 12). Methods of producing fusion proteins utilizing this affinity tag for purification are described in U.S. Pat. No. 5,011,912.

A still further component of the fusion protein can be a signal sequence or leader sequence. These sequences are generally utilized to allow for secretion of the fusion protein from the host cell during expression and are also known as a leader sequence, prepro sequence or pre sequence. The secretory signal sequence may be that of the heterologous protein being produced, if it has such a sequence, or may be derived from another secreted protein (e.g., t-PA) or synthesized de novo. The secretory signal sequence is operably linked to fusion protein DNA sequence, i.e., the two sequences are joined in the correct reading frame and positioned to direct the newly sythesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

### Preparation of Polynucleotides Encoding VASP-Heterologous Fusion Proteins

The nucleic acid compositions of the present invention find use in the preparation of all or a portion of the VASP-Heterologous fusion proteins, as described above. The subject polynucleotides (including cDNA or the full-length gene) can be used to express a partial or complete gene product. Constructs comprising the subject polynucleotides can be generated synthetically. Alternatively, single-step assembly of a gene and entire plasmid from large numbers of oligodeoxyribonucleotides is described by, e.g., Stemmer et al., Gene (Amsterdam) (1995) 164(1):49-53. In this method, assembly PCR (the synthesis of long DNA sequences from large numbers of oligodeoxyribonucleotides (oligos)) is described. The method is derived from DNA shuffling (Stemmer, Nature (1994) 370:389-391), and does not rely on DNA ligase, but instead relies on DNA polymerase to build increasingly longer DNA fragments during the assembly process. Appropriate polynucleotide constructs are purified using standard recombinant DNA techniques as described in, for example, Sambrook et al., Molecular Cloning: A Laboratory Manual, 2nd Ed., (1989) Cold Spring Harbor Press, Cold Spring Harbor, N.Y., and under current regulations described in United States Dept. of HHS, National Institute of Health (NIH) Guidelines for Recombinant DNA Research.

Polynucleotide molecules comprising a polynucleotide sequence provided herein are propagated by placing the molecule in a vector. Viral and non-viral vectors are used, including plasmids. The choice of plasmid will depend on the type of cell in which propagation is desired and the purpose of propagation. Certain vectors are useful for amplifying and making large amounts of the desired DNA sequence. Other vectors are suitable for expression in cells in culture. Still other vectors are suitable for transfer and expression in cells in a whole animal or person. The choice of appropriate vector is well within the skill of the art. Many such vectors are available commercially. The partial or full-length polynucleotide is inserted into a vector typically by means of DNA ligase attachment to a cleaved restriction enzyme site in the vector. Alternatively, the desired nucleotide sequence can be inserted by homologous recombination in vivo. Typically this is accomplished by attaching regions of homology to the vector on the flanks of the desired nucleotide sequence. Regions of homology are added by ligation of oligonucleotides, or by polymerase chain reaction using primers comprising both the region of homology and a portion of the desired nucleotide sequence, for example.

For expression, an expression cassette or system may be employed. The gene product encoded by a polynucleotide of the invention is expressed in any convenient expression system, including, for example, bacterial, yeast, insect, amphibian and mammalian systems. Suitable vectors and host cells are described in U.S. Pat. No. 5,654,173. In the expression vector, the heterologous protein encoding polynucleotide (such as the extracellular domain of zB7R1 (SEQ ID NO:19) or the extracellular domain of B7H1) is linked to a regulatory sequence as appropriate to obtain the desired expression properties. These can include promoters (attached either at the 5′ end of the sense strand or at the 3′ end of the antisense strand), enhancers, terminators, operators, repressors, and inducers. The promoters can be regulated or constitutive. In some situations it may be desirable to use conditionally active promoters, such as tissue-specific or developmental stage-specific promoters. These are linked to the desired nucleotide sequence using the techniques described above for linkage to vectors. Any techniques known in the art can be used. In other words, the expression vector will provide a transcriptional and translational initiation region, which may be inducible or constitutive, where the coding region is operably linked under the transcriptional control of the transcriptional initiation region, and a transcriptional and translational termination region. These control regions may be native to the DNA encoding the VASP-heterologous fusion protein, or may be derived from exogenous sources.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region. Of particular interest is the use of sequences that allow for the expression of functional epitopes or domains, usually at least about 8 amino acids in length, more usually at least about 15 amino acids in length, to about 25 amino acids, and up to the complete open reading frame of the gene. After introduction of the DNA, the cells containing the construct may be selected by means of a selectable marker, the cells expanded and then used for expression.

VASP-Heterologous fusion proteins may be expressed in prokaryotes or eukaryotes in accordance with conventional ways, depending upon the purpose for expression. For large scale production of the protein, a unicellular organism, such as E. coli, B. subtilis, S. cerevisiae, insect cells in combination with baculovirus vectors, or cells of a higher organism such as vertebrates, particularly mammals, e.g. COS 7 cells, HEK 293, CHO, Xenopus Oocytes, etc., may be used as the expression host cells. In some situations, it is desirable to express a polymorphic VASP nucleic acid molecule in eukaryotic cells, where the polymorphic VASP protein will benefit from native folding and post-translational modifications. Small peptides can also be synthesized in the laboratory. Polypeptides that are subsets of the complete VASP sequence may be used to identify and investigate parts of the protein important for function.

Specific expression systems of interest include bacterial, yeast, insect cell and mammalian cell derived expression systems. Representative systems from each of these categories is are provided below:

Bacteria. Expression systems in bacteria include those described in Chang et al., Nature (1978) 275:615; Goeddel et al., Nature (1979) 281:544; Goeddel et al., Nucleic Acids Res. (1980) 8:4057; EP 0 036,776; U.S. Pat. No. 4,551,433; DeBoer et al., Proc. Natl. Acad. Sci. (USA) (1983) 80:21-25; and Siebenlist et al., Cell (1980) 20:269.

Yeast. Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142; Kunze et al., J. Basic Microbiol. (1985)25:141; Gleeson et al., J. Gen. Microbiol. (1986) 132:3459; Roggenkamp et al., Mol. Gen. Genet. (1986) 202:302; Das et al., J. Bacteriol. (1984) 158:1165; De Louvencourt et al., J. Bacteriol. (1983) 154:737; Van den Berg et al., Bio/Technology (1990)8:135; Kunze et al., J. Basic Microbiol. (1985)25:141; Cregg et al., Mol. Cell. Biol. (1985) 5:3376; U.S. Pat. Nos. 4,837,148 and 4,929,555; Beach and Nurse, Nature (1981) 300:706; Davidow et al., Curr. Genet. (1985) 10:380; Gaillardin et al., Curr. Genet. (1985) 10:49; Ballance et al., Biochem. Biophys. Res. Commun. (1983) 112:284-289; Tilburn et al., Gene (1983) 26:205-221; Yelton et al., Proc. Natl. Acad. Sci. (USA) (1984) 81:1470-1474; Kelly and Hynes, EMBO J. (1985) 4:475479; EP 0 244,234; and WO 91/00357.

Insect Cells. Expression of heterologous genes in insects is accomplished as described in U.S. Pat. No. 4,745,051; Friesen et al., “The Regulation of Baculovirus Gene Expression”, in: The Molecular Biology Of Baculoviruses (1986) (W. Doerfler, ed.); EP 0 127,839; EP 0 155,476; and Vlak et al., J. Gen. Virol. (1988) 69:765-776; Miller et al., Ann. Rev. Microbiol. (1988) 42:177; Carbonell et al., Gene (1988) 73:409; Maeda et al., Nature (1985) 315:592-594; Lebacq-Verheyden et al., Mol. Cell. Biol. (1988) 8:3129; Smith et al., Proc. Natl. Acad. Sci. (USA) (1985) 82:8844; Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Generic Engineering (1986) 8:277-279, and Maeda et al., Nature (1985) 315:592-594.

Mammalian Cells. Mammalian expression is accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. Pat. No. RE 30,985.

When any of the above host cells, or other appropriate host cells or organisms, are used to replicate and/or express the polynucleotides or nucleic acids of the invention, the resulting replicated nucleic acid, RNA, expressed protein or polypeptide, is within the scope of the invention as a product of the host cell or organism. The product is recovered by any appropriate means known in the art.

Once the gene corresponding to a selected polynucleotide is identified, its expression can be regulated-in the cell to which the gene is native. For example, an endogenous gene of a cell can be regulated by an exogenous regulatory sequence inserted into the genome of the cell at location sufficient to at least enhance expressed of the gene in the cell. The regulatory sequence may be designed to integrate into the genome via homologous recombination, as disclosed in U.S. Pat. Nos. 5,641,670 and 5,733,761, the disclosures of which are herein incorporated by reference, or may be designed to integrate into the genome via non-homologous recombination, as described in WO 99/15650, the disclosure of which is herein incorporated by reference.

### Vectors and Host Cells Comprising the Polynucleotides of the Invention

The invention further provides recombinant vectors and host cells comprising polynucleotides of the invention. In general, recombinant vectors and host cells of the invention are isolated; however, a host cell comprising a polynucleotide of the invention may be part of a genetically modified animal.

Recombinant vectors. The present invention further provides recombinant vectors (“constructs”) comprising a polynucleotide of the invention. Recombinant vectors include vectors used for propagation of a polynucleotide of the invention, and expression vectors. Vectors useful for introduction of the polynucleotide include plasmids and viral vectors, e.g. retroviral-based vectors, adenovirus vectors, etc. that are maintained transiently or stably in mammalian cells. A wide variety of vectors can be employed for transfection and/or integration of the gene into the genome of the cells. Alternatively, micro-injection may be employed, fusion, or the like for introduction of genes into a suitable host cell.

Expression vectors generally have convenient restriction sites located near the promoter sequence to provide for the insertion of nucleic acid sequences encoding heterologous proteins. A selectable marker operative in the expression host may be present. Expression vectors may be used for the production of fusion proteins, where the exogenous fusion peptide provides additional functionality, i.e. increased protein synthesis, stability, reactivity with defined antisera, an enzyme marker, e.g. β-galactosidase, etc.

Expression cassettes may be prepared comprising a transcription initiation region, the gene or fragment thereof, and a transcriptional termination region. Of particular interest is the use of sequences that allow for the expression of functional epitopes or domains, usually at least about 8 amino acids in length, more usually at least about 15 amino acids in length, at least about 25 amino acids, at least about 45 amino acids, and up to the complete open reading frame of the gene. After introduction of the DNA, the cells containing the construct may be selected by means of a selectable marker, the cells expanded and then used for expression.

The expression cassettes may be introduced into a variety of vectors, e.g. plasmid, BAC, YAC, bacteriophage such as lambda, P1, M13, etc., animal or plant viruses, and the like, where the vectors are normally characterized by the ability to provide selection of cells comprising the expression vectors. The vectors may provide for extrachromosomal maintenance, particularly as plasmids or viruses, or for integration into the host chromosome. Where extrachromosomal maintenance is desired, an origin sequence is provided for the replication of the plasmid, which may be low- or high copy-number. A wide variety of markers are available for selection, particularly those which protect against toxins, more particularly against antibiotics. The particular marker that is chosen is selected in accordance with the nature of the host, where in some cases, complementation may be employed with auxotrophic hosts. Introduction of the DNA construct may use any convenient method, e.g. conjugation, bacterial transformation, calcium-precipitated DNA, electroporation, fusion, transfection, infection with viral vectors, biolistics, etc.

Genetically Modified Cells. The present invention further provides host cells, which may be isolated host cells, comprising polymorphic VASP nucleic acid molecules of the invention. Suitable host cells include prokaryotes such as E. coli, B. subtilis, eukaryotes, including insect cells in combination with baculovirus vectors, yeast cells, such as Saccharomyces cerevisiae, or cells of a higher organism such as vertebrates, including amphibians (e.g., Xenopus laevis oocytes), and mammals, particularly humans, e.g. COS cells, CHO cells, HEK293 cells, and the like, may be used as the host cells. Host cells can be used for the purposes of propagating a polymorphic VASP nucleic acid molecule, for production of a polymorphic VASP polypeptide, or in cell-based methods for identifying agents which modulate a level of VASP mRNA and/or protein and/or biological activity in a cell.

Primary or cloned cells and cell lines may be modified by the introduction of vectors comprising a DNA encoding the VASP-heterologous fusion protein polymorphism(s). The isolated polymorphic VASP nucleic acid molecule may comprise one or more variant sequences, e.g., a haplotype of commonly occurring combinations. In one embodiment of the invention, a panel of two or more genetically modified cell lines, each cell line comprising a VASP polymorphism, are provided for substrate and/or expression assays. The panel may further comprise cells genetically modified with other genetic sequences, including polymorphisms, particularly other sequences of interest for pharmacogenetic screening, e.g. other genes/gene mutations associated with obesity, a number of which are known in the art.

Transgenic animals. The subject nucleic acids can be used to generate genetically modified non-human animals or site specific gene modifications in cell lines. The term “transgenic” is intended to encompass genetically modified animals having the addition of DNA encoding the VASP-heterologous fusion protein or having an exogenous DNA encoding the VASP-heterologous fusion protein that is stably transmitted in the host cells. Transgenic animals may be made through homologous recombination. Alternatively, a nucleic acid construct is randomly integrated into the genome. Vectors for stable integration include plasmids, retroviruses and other animal viruses, YACs, and the like. Of interest are transgenic mammals, e.g. cows, pigs, goats, horses, etc., and particularly rodents, e.g. rats, mice, etc.

DNA constructs for homologous recombination will comprise at least a portion of the DNA encoding the VASP-heterologous fusion protein and will include regions of homology to the target locus. Conveniently, markers for positive and negative selection are included. Methods for generating cells having targeted gene modifications through homologous recombination are known in the-art. For various techniques for transfecting mammalian cells, see Known et al. (1990) Methods in Enzymology 185:527-537.

For embryonic stem (ES) cells, an ES cell line may be employed, or ES cells may be obtained freshly from a host, e.g. mouse, rat, guinea pig, etc. Such cells are grown on an appropriate fibroblast-feeder layer or grown in the presence of leukemia inhibiting factor (LIF). When ES cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination. Those colonies that show homologous recombination may then be used for embryo manipulation and blastocyst injection. Blastocysts are obtained from. 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting litters screened for mutant cells having the construct. By providing for a different phenotype of the blastocyst and the ES cells, chimeric progeny can be readily detected. The chimeric animals are screened for the presence of the DNA encoding the VASP-heterologous fusion protein and males and females having the modification are mated to produce homozygous progeny. The transgenic animals may be any non-human mammal, such as laboratory animals, domestic animals, etc. The transgenic animals may be used to determine the effect of a candidate drug in an in vivo environment.

### Production of Homo- or Hetero-Tetrameric Proteins Utilizing VASP Constructs

The present invention is a method of preparing a soluble, homo- or hetero-trimeric protein by culturing a host cell transformed or transfected with at least one or up to four different expression vectors encoding a fusion protein comprising a VASP domain and a heterologous protein. In order to produce a biologically functioning protein, the four VASP domains preferentially form a homo- or hetero-tetramers. The culturing can also occur in the same host cell, if efficient production can be maintained, and homo- or hetero-tetrameric proteins are then isolated from the medium. Ideally, the four heterologous proteins are differentially labeled with various tag sequences (i.e., His tag, FLAG tag, and Glu-Glu tag) to allow analysis of the composition or purification of the resulting molecules. Alternatively, the four components can be produced separately and combined in deliberate ratios to result in the hetero-tetrameric molecules desired. The VASP domains utilized in making these hetero-trimeric molecules can be the same or different and the fusion protein(s) can further comprise a linker sequence. In one particular embodiment, the heterologous protein used to form the homo-tetrameric protein is the soluble domain of B7H1. In another particular embodiment, the heterologous protein used to form the homo-tetrameric protein is the soluble domain of zB7R1.

One result of the use of the VASP tetramerization domain of the present invention is the ability to increase the affinity and avidity of the heterologous protein for its ligand or binding partner through the formation of the terameric form. By avidity, it is meant the strength of binding of multiple molecules to a larger molecule, a situation exemplified but not limited to the binding of a complex antigen by an antibody. Such a characteristic would be improved or formed for many heterologous proteins, for example, by the formation of multiple binding sites for its ligand or ligands through the tetramerization of the heterologous receptor using the VASP domain. By affinity, it is meant the strength of binding of a simple receptor-ligand system. Such a characteristic would be improved for a subset of heterologous proteins using the tetramerization domain of the present invention, for example, by forming a binding site with better binding characteristics for a single ligand through the tetramerization of the receptor. Avidity and affinity can be measured using standard assays well known to one of ordinary skill, for example, the methods described in Example 3. An improvement in affinity or avidity occurs when the affinity or avidity value (for example, affinity constant or Kₐ) for the tetramerization domain-heterologous protein fusion and its ligand is higher than for the heterologous protein alone and its ligand. An alternative means of measuring these characteristics is the equilibrium constant (K_{d}) where a decrease would be observed with the improvement in affinity or avidity using the VASP tetermerization domain of the present invention.

### Biological Activity of the VASP-Heterologous Fusion Proteins

Biological activity of recombinant VASP-heterologous fusion proteins is mediated by binding of the recombinant fusion protein to a cognate molecule, such as a receptor or cross-receptor. A cognate molecule is defined as a molecule which binds the recombinant fusion protein in a non-covalent interaction based upon the proper conformation of the recombinant fusion protein and the cognate molecule. For example, for a recombinant fusion protein comprising an extracellular region of a receptor, the cognate molecule comprises a ligand which binds the extracellular region of the receptor. Conversely, for a recombinant soluble fusion protein comprising a ligand, the cognate molecule comprises a receptor (or binding protein) which binds the ligand.

Binding of a recombinant fusion protein to a cognate molecule is a marker for biological activity. Such binding activity may be determined, for example, by competition for binding to the binding domain of the cognate molecule (i.e. competitive binding assays). One configuration of a competitive binding assay for a recombinant fusion protein comprising a ligand uses a radiolabeled, soluble receptor, and intact cells expressing a native form of the ligand. Similarly, a competitive assay for a recombinant fusion protein comprising a receptor uses a radiolabeled, soluble ligand, and intact cells expressing a native form of the receptor. Such an assay is described in Example 3. Instead of intact cells expressing a native form of the cognate molecule, one could substitute purified cognate molecule bound to a solid phase. Competitive binding assays can be performed using standard methodology. Qualitative or semi-quantitative results can be obtained by competitive autoradiographic plate binding assays, or fluorescence activated cell sorting, or Scatchard plots may be utilized to generate quantitative results.

Biological activity may also be measured using bioassays that are known in the art, such as a cell proliferation assay. An exemplary bioassay is described in Example 4. The type of cell proliferation assay used will depend upon the recombinant soluble fusion protein. For example, a bioassay for a recombinant soluble fusion protein that in its native form acts upon T cells will utilize purified T cells obtained by methods that are known in the art. Such bioassays include costimulation assays in which the purified T cells are incubated in the presence of the recombinant soluble fusion protein and a suboptimal level of a mitogen such as Con A or PHA. Similarly, purified B cells will be used for a recombinant soluble fusion protein that in its native form acts upon B cells. Other types of cells may also be selected based upon the cell type upon which the native form of the recombinant soluble fusion protein acts. Proliferation is determined by measuring the incorporation of a radiolabeled substance, such as ³H thymidine, according to standard methods.

Yet another type assay for determining biological activity is induction of secretion of secondary molecules. For example, certain proteins induce secretion of cytokines by T cells. T cells are purified and stimulated with a recombinant soluble fusion protein under the conditions required to induce cytokine secretion (for example, in the presence of a comitogen). Induction of cytokine secretion is determined by bioassay, measuring the proliferation of a cytokine dependent cell line. Similarly, induction of immunoglobulin secretion is determined by measuring the amount of immunoglobulin secreted by purified B cells stimulated with a recombinant soluble fusion protein that acts on B cells in its native form, using a quantitative (or semi-quantitative) assay such as an enzyme immunoassay.

If the binding partner for a particular heterologous protein is unknown, the VASP-fusion protein can be used in a binding assay to seek out that binding partner. One method of doing this, called a secretion trap assay, is described in Example 5, although other methods of using a VASP-fusion protein to identify binding partners are well known to one of ordinary skill.

### Treatment Methods

For pharmaceutical use, the fusion proteins of the present invention are formulated for parenteral, particularly intravenous or subcutaneous, administration according to conventional methods. Intravenous administration will be by bolus injection or infusion over a typical period of one to several hours. In general, pharmaceutical formulations will include a VASP-heterologous fusion protein in combination with a pharmaceutically acceptable vehicle, such as saline, buffered saline, 5% dextrose in water or the like. Formulations may further include one or more excipients, preservatives, solubilizers, buffering agents, albumin to prevent protein loss on vial surfaces, etc. Methods of formulation are well known in the art and are disclosed, for example, in Remington's Pharmaceutical Sciences, Gennaro, ed., Mack Publishing Co., Easton Pa., 1990, which is incorporated herein by reference. Therapeutic doses will generally be in the range of 0.1 to 100 μg/kg of patient weight per day, preferably 0.5-20 μg/kg per day, with the exact dose determined by the clinician according to accepted standards, taking into account the nature and severity of the condition to be treated, patient traits, etc. Determination of dose is within the level of ordinary skill in the art. The proteins may be administered for acute treatment, over one week or less, often over a period of one to three days or may be used in chronic treatment, over several months or years. In general, a therapeutically effective amount of VASP-heterologous fusion protein is an amount sufficient to produce a clinically significant change in the symptoms characteristics of the lack of heterologous protein function. Alternatively, if the VASP-heterologous fusion protein is to act as an antagonist, a therapeutically effective amount is that which produces a clinically significant change in symptoms characteristic of an over-abundance of heterologous protein function.

### VASP Fusions to the Extracellular Domains of the Following Receptors.

Using essentially the methods described in the Examples that follow VASP fusion proteins have been made to the extracellular domains of these B7 family members: pb7H1 (see Examples), pb7H3, pb7H4, pb7DC, pG6B, pNKp30, pNFAM, pHHLA2, and pPVR as well as murine pNFAM. The resulting proteins were expressed well in CHO cells or BHK cells as tetrameric oligomers.

The invention is further illustrated by the following non-limiting examples.

### Example 1

### Cloning and Construction of VASP Expression Vector

Human vasodialator-activated phosphoprotein (VASP) is described by Kühnel, et al., (2004) Proc. Nat'l. Acad. Sci. 101: 17027. VASP nucleotide and amino acid sequences are provided as SEQ ID NOS. 1 and 2. Two overlapping oligonucleotides, which encoded both sense and antisense strands of the tetramerization domain of human VASP protein, were synthesized by solid phased synthesis: 5′ ACGCTTCCGT AGATCTGGTT CCGGAGGCTC CGGTGGCTCC GACCTACAGA GGGTGAAACA GGAGCTTCTG GAAGAGGTGA AGAAGGAATT GCAGAAGTGA AAG 3′ (zc50629, SEQ ID NO:3); 5′ AAGGCGCGCC TCTAGATCAG TGATGGTGAT GGTGATGGCC ACCGGAACCC CTCAGCTCCT GGACGAAGGC TTCAATGATT TCCTCTTTCA CTTTCTGCAA TTC 3′ (ZC 50630, SEQ ID NO:4). The oligonucleotides zc50629 and zc50630 were annealed at 55° C., and amplified by PCR with the olignucleotide primers zc50955 (5′ CTCAGCCAGG AAATCCATGC CGAGTTGAGA CGCTTCCGTA GATCTGG 3′) (SEQ ID NO:5) and zc50956 (5′ GGGGTGGGGT ACAACCCCAG AGCTGTTTTA AGGCGCGCCT CTAGATC 3′) (SEQ ID NO:6).

The amplified DNA was fractionated on 1.5% agarose gel and then isolated using a Qiagen gel isolation kit according to manufacturer's protocol (Qiagen, Valiencia, Calif.). The isolated DNA was inserted into BglII cleaved pzmp21 vector by yeast recombination. DNA sequencing confirmed the expected sequence of the vector, which was designated pzmp21VASP-His₆.

### Construction of an Expression Vector for Tetrameric B7H1VASP-His₆

The extracellular domain of B7H1 was amplified by PCR with oligonucleotide primers zc51310 (5′ CCACAGGTGTCCAGGGAATTCGCAAGATGAGGATATTTGCTGTC 3′) (SEQ ID NO:7) and zc51312 (5′ CTCCGGAACCAGATCTTTCATTTGGAGGATGTGC 3′) (SEQ ID NO:8). The amplified DNA was fractionated on 1.5% agarose gel and then isolated using a Qiagen gel isolation kit according to manufacturer's protocol (Qiagen, Valiencia, Calif.). The isolated DNA was inserted into BglII and EcoR1 cleaved pzmp21VASP-His₆ vector by in fusion according to the manufacturers instruction (BD Biosciences, San Diego, Calif.). DNA sequencing confirmed the expected sequence of the vector, which was designated pzmp21B7H1VASP-His₆, the B7H1-VASP-His₆ portion is disclosed herein as SEQ ID NO: 9, with the resulting polypeptide sequence being SEQ ID NO: 10.

This vector includes the coding sequence for the B7H1 extracellular domain comprising amino acids 1 to 239 of the full length gene (amino acids 1 to 239 of SEQ ID NO:13) (this includes the gene's native signal sequence of the first 18 amino acids), the flexible linker GSGG (amino acids 1 to 4 of SEQ ID NO:2 or SEQ ID NO: 11), the VASP tetramerization domain (amino acids 5 to 38 of SEQ ID NO: 2), the flexible linker GSGG (amino acids 39 to 42 of SEQ ID NO: 2 or SEQ ID NO:11), and the His6 tag amino acid residues (amino acids 43 to 48 of SEQ ID NO: 2 or SEQ ID NO: 12).

### Example 2

### Expression and Purification of B7H1VASP-HIS₆

The pzmp21B7H1VASP-His₆ vector was transfected into BHK570 cells using Lipofectamine 2000 according to manufacturer's protocol (Invitrogen, Carlsbad, Calif.) and the cultures were selected for transfectants resistance to 10 μM methotrexate. Resistant colonies were transferred to tissue culture dishes, expanded and analyzed for secretion of B7H1VASP-His₆ by western blot analysis with Anti-His (C-terminal) Antibody (Invitrogen, Carlsbad, Calif.). The resulting cell line, BHK.B7H1VASP-His₆.2, was expanded.

A) Purification of B7H1VASP-His₆ from BHK Cells

The purification was performed at 4° C. About 2 L of conditioned media from BHK:B7H1VASP-His₆.2 was concentrated to 0.2 L using Pellicon-2 5k filters (Millipore, Bedford, Mass.), then buffer-exchanged tenfold with 20 mM NaPO₄, 0.5M NaCl, 15 mM Imidazole, pH 7.5. The final 0.2 L sample was passed-through a 0.2 mm filter (Millipore, Bedford, Mass.).

A Talon (BD Biosciences, San Diego, Calif.) column with a 20 mL bed-volume was packed and equilibrated with 20 mM NaPi, 15 mM Imidazole, 0.5 M NaCl, pH 7.5. The media was loaded onto the column at a flow-rate of 0.2-0.4 mL/min then washed with 5-6 CV of the equilibration buffer. B7H1VASP-His₆ was eluted from the column with 20 mM NaPO₄, 0.5 M NaCl, 0.5 M Imidazole, pH 7.5 at a flow-rate of 4 mL/min. 10 mL fractions were collected and analyzed for the presence of B7H1VASP-His₆ by Coomassie-stained SDS-PAGE.

A combined pool of Talon eluates obtained from three identical runs as described above was concentrated from 60 mL to 3 mL using an Amicon Ultra 5k centrifugal filter (Millipore, Bedford, Mass.). A Superdex 200 column with a bed-volume of 318 mL was equilibrated with 50 mM NaPi, 110 mM NaCl, pH 7.3, and the 3 mL sample was injected into the column at a flow-rate of 0.5 mL/min. Two 280 nm absorbance peaks were observed eluting from the column, one at 0.38 CV and the other at 0.44 CV. The fractions eluting around 0.44 CV, believed to contain tetrameric B7H1VASP-His₆, were pooled and concentrated, sterile-filtered through a 0.2 mm Acrodisc filter (Pall Corporation, East Hills, N.Y.), and stored at −80° C. Concentration of the final sample was determined by BCA (Pierce, Rockford, Ill.).

### B) SEC-MALS Analysis of B7H1VASP-CH₆

The purpose of size exclusion chromatography (SEC) is to separate molecules on the basis of size for estimation of molecular weight (M_{W}). If static light scattering detection is added to a SEC system, absolute measurements of molecular weight can be made. This is possible because the intensity of light scattered by the analyte is directly proportional to its mass and concentration, and is completely independent of SEC elution position, conformation or interaction with the column matrix. Additionally, by combining SEC, multi-angle laser light scattering (MALS) and refractive index detection (RI), the molecular mass, association state, and degree of glycosylation can be determined The limit of accuracy of these measurements for a sample that is monodisperse with respect to M_{W} is ±2%.

The molecular mass of monomeric B7H1VASP-CH₆, predicted from primary amino acid sequence is 31 kDa. The predicted molecular mass of tetrameric B7H1VASP-CH₆ would be 124 Kda. The measured molecular mass of B7H1VASP-CH₆ measured by SEC-MALS was 155 KDa. Subtraction of 35 Kda of molelcular mass due to carbohydrate leaves 120 KDa as the mass of the core protein, consistent with a tetrameric state in solution.

### Example 3

### Test of Binding Activity of ¹²⁵I-VASP-B7H1 Fusion Protein to Cell Lines

### A) Saturation Binding

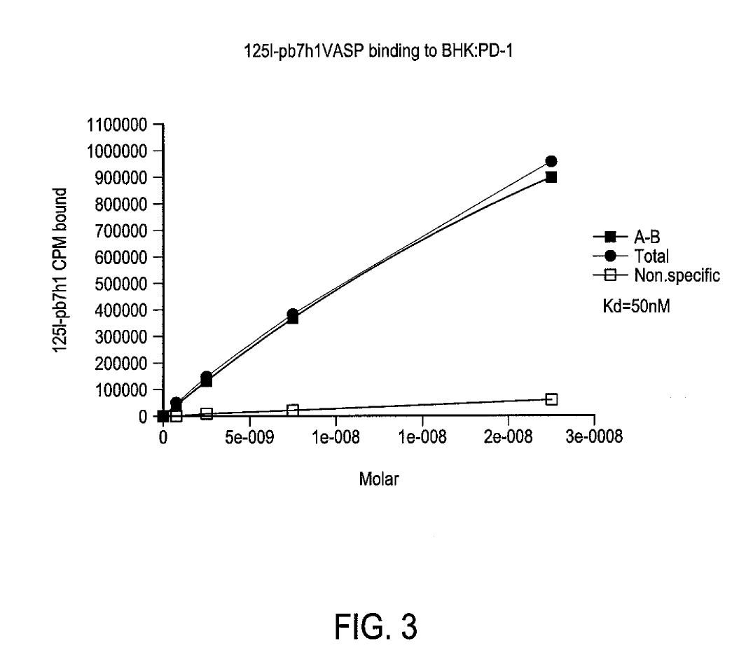

25 mg of purified B7H1VASP-His₆ was labeled with 2 mCi ¹²⁵I using IODO-TUBES (Pierce, Rockford, Ill.) according to manufacturer's instructions. This labeled protein was used to asses binding to transfected BHK 570 cells expressing PD-1, the ligand for B7H1 (ref), with untransfected BHK-570 cells as control. 1×10⁵ cells were plated in 24 well dishes and cultured for two days. Concentrations of ¹²⁵I-B7H1VASP-His₆, from 22.5 nM to 10.3 pM, with or without 100 fold excess of unlabeled B7H1VASP-His₆, was added to triplicate wells of cells. The binding reactions were incubated for one hour on ice, and then the cells were washed 3× with ice cold binding buffer. Bound proteins were extracted with 1 M NaOH and quantitated on the COBRAII Auto-gamma counter (Packard Instruments Co., Meriden, Conn.) Analysis of the binding was done using GraphPad, Prism 4 (GraphPad Software, Inc., San Diego, Calif.). The results of this experiment are reported in FIG. 3.

Saturation binding and inhibition by unlabeled protein revealed high affinity (Kd 50 nM) binding of tetrameric B7H1VASP-His₆ to cell surface PD-1. This is 10 fold higher affinity than that reported for B7H1IgG (Freeman et al., (2000) J. Exp. Med. 192: 1027).

### B) Binding Specificity

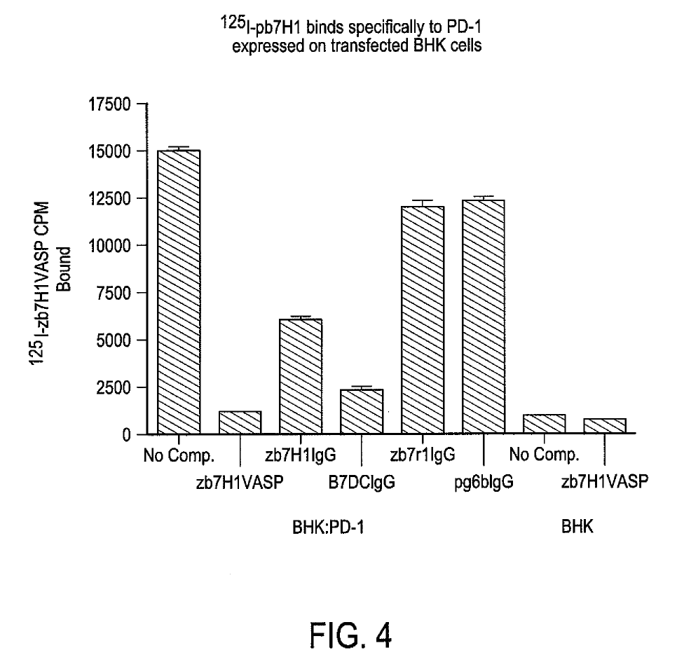

1×10⁵ cells were plated in 24 well dishes and cultured for two days. 250 pM of ¹²⁵I-B7H1VASP-His₆ with or without 100 fold excess of unlabeled B7H1VASP-His₆, B7H1IgG, B7DCIgG (R & D Systems, Minneapolis, Minn.), zB7R1IgG, or pG6BIgG was added to triplicate wells of cells. The binding reactions were incubated for one hour on ice, and then the cells were washed 3× with ice cold binding buffer. Bound proteins were extracted with 1 M NaOH and quantitated on the COBRAII Auto-gamma counter (Packard Instruments Co., Meriden, Conn.) Analysis of the binding was done using GraphPad, Prism 4 (GraphPad Software, Inc., San Diego, Calif.). ¹²⁵I-B7H1VASP-His₆ binds only to transfected BHK cells expressing PD-1 and not to untransfected cells. The specificity of the interaction of zB7H1VASP is demonstrated by the ability of PD-1 ligands to inhibit binding, while other B7 family members, that do not interact with PD-1, do not affect binding. A graph showing the results of this experiment is in FIG. 4.

### C) Competition of ¹²⁵I-B7H1VASP-His₆ Binding by B7H1VASP-His₆ or B7H1IgG.

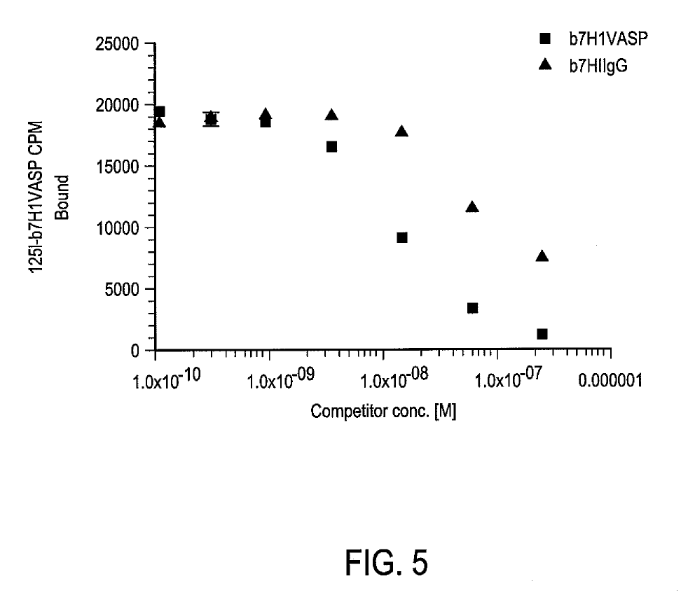

1×10⁵ cells were plated in 24 well dishes and cultured for two days. 250 pM of ¹²⁵I-B7H1VASP-His₆, without or with increasing concentration of unlabeled B7H1VASP-His₆, or B7H1IgG (R & D Systems, Minneapolis, Minn.), was added to triplicate wells of cells. The binding reactions were incubated for one hour on ice, and then the cells were washed 3× with ice cold binding buffer. Bound proteins were extracted with 1 M NaOH and quantitated on the COBRAII Auto-gamma counter (Packard Instruments Co., Meriden, Conn.) Analysis of the binding was done using GraphPad, Prism 4 (GraphPad Software, Inc., San Diego, Calif.). The 10 fold greater affinity of B7H1VASP, as compared to B7H1IgG, is demonstrated by the shift in competition for ¹²⁵I-B7H1VASP-His₆ binding to lower concentration. A graph illustrating this result is in FIG. 5.

### Example 4

### Biological Activity of the B7H1-VASP Fusion Protein

T-cells are isolated from peripheral blood by negative selection (Mitenyi Biotec, Auburn, Calif.). T-cells are plated into each well of a 96 well dish that had been pre-coated with anti-CD3 (BD Bioscience, San Diego, Calif.). Anti-CD28 (BD Bioscience, San Diego, Calif.), and increasing concentration of B7H1-VASP are added to appropriate wells. The cultures are incubated at 37° C. for 4 days and then labeled overnight with 1 μCi [³H] thymidine per well. Proliferation is measured as [³H] thymidine incorporated, and culture cytokine content is quantitated using Luminex (Austen, Tex.). B7H1VASP is expected to potently inhibit both T-cell proliferation and cytokine release (Dong et al., Nature Med. 5: 1365-1369, 1999).

### Example 5

### Use of VASP-Protein Fusion to Screen for Ligands

A) Screening of the cDNA Library:

A secretion trap assay is used to pair VASP-protein fusions to putative ligands or binding partners. A soluble VASP fusion protein that has been biotinylated is used as a binding reagent in a secretion trap assay. A cDNA library from cells of interest, for example, stimulated mouse bone marrow (mBMDC) is transiently transfected into COS cells in pools of clones. Commonly, about 800 clones are produced for the initial transfection. The binding of the biotinylated VASP-protein fusion to transfected COS cells is carried out using the secretion trap assay described below. Positive binding is seen in a subset of the pools screened. One of these pools is selected and electroporated into a bacterial host such as DH10B. 400 single colonies are picked into 1.2 mls LB+100 ug/ml ampicillin in deep well 96-well blocks, grown overnight followed by DNA isolation from each plate. After transfection and secretion trap probe, positive wells are identified from this breakdown and submitted to sequencing and are identified through comparison to known sequences. The purified cDNA is transfected and probed with biotinylated VASP-protein fusion along with additional controls to verify that the identified protein specifically and reproducibly binds to the VASP-fusion protein but not other VASP chimeras.

### B) COS Cell Transfections

The COS cell transfection is performed as follows: Mix lug pooled DNA in 25 ul of serum free DMEM media (500 mls DMEM with 5 mls non-essential amino acids) and 1 ul Cosfectin™ in 25 ul serum free DMEM media. The diluted DNA and cosfectin are then combined followed by incubating at room temperature for 30 minutes. Add this 50 ul mixture onto 8.5×10⁵ COS cells/well that have been plated on the previous day in 12-well tissue culture plates and incubate overnight at 37° C.

### C) Secretion Trap Assay

The secretion trap is performed as follows: Media is aspirated from the wells and then the cells are fixed for 15 minutes with 1.8% formaldehyde in PBS. Cells are then washed with TNT (0.1M Tris-HCL, 0.15M NaCl, and 0.05% Tween-20 in H₂O), and permeabilized with 0.1% Triton-X in PBS for 15 minutes, and again washed with TNT. Cells are blocked for 1 hour with TNB (0.1M Tris-HCL, 0.15M NaCl and 0.5% Blocking Reagent (NEN Renaissance TSA-Direct Kit) in H₂O), and washed again with TNT. The cells are incubated for 1 hour with 2 μg/ml soluble biotinylated VASP-fusion protein. Cells are then washed with TNT. Cells are fixed a second time for 15 minutes with 1.8% formaldehyde in PBS. After washing with TNT, cells are incubated for another hour with 1:1000 diluted streptavidin HRP. Again cells are washed with TNT.

Positive binding is detected with fluorescein tyramide reagent diluted 1:50 in dilution buffer (NEN kit) and incubated for 5 minutes, and washed with TNT. Cells are preserved with Vectashield Mounting Media (Vector Labs Burlingame, Calif.) diluted 1:5 in TNT. Cells are visualized using a FITC filter on fluorescent microscope.

### Example 6

### Use of VASP-zB7R1 Fusion Protein to Screen for Ligands

zB7R1VASP fusion protein was made as described in Examples 1-5 for B7H1VASP. This protein was then used to screen for its corresponding ligand as described below.

A) Screening of the mBMDC Library:

A secretion trap assay was used to pair mzB7R1 to mCD155 (PVR). The soluble mzB7R1/Vasp fusion protein that had been biotinylated was used as a binding reagent in a secretion trap assay. A pZP-7NX cDNA library from stimulated mouse bone marrow (mBMDC) was transiently transfected into COS cells in pools of 800 clones. The binding of mzB7R1/Vasp-biotin to transfected COS cells was carried out using the secretion trap assay described below. Positive binding was seen in 26 of 72 pools screened. One of these pools was selected and electroporated into DH10B. 400 single colonies were picked into 1.2 mls LB+100 ug/ml ampicillin in deep well 96-well blocks, grown overnight followed by DNA isolation from each plate. After transfection and secretion trap probe, a single positive well was identified from this breakdown and submitted to sequencing and was identified as being mCD155. This purified cDNA was transfected and probed with mB7R1/Vasp-biotin along with additional controls to verify that mCD155 specifically and reproducibly bound mB7R1/Vasp-biotin but not other vasp chimeras.

### B) COS Cell Transfections

The COS cell transfection was performed as follows: Mix lug pooled DNA in 25 ul of serum free DMEM media (500 mls DMEM with 5 mls non-essential amino acids) and 1 ul Cosfectin™ in 25 ul serum free DMEM media. The diluted DNA and cosfectin are then combined followed by incubating at room temperature for 30 minutes. Add this 50 ul mixture onto 8.5×10⁵ COS cells/well that had been plated on the previous day in 12-well tissue culture plates and incubate overnight at 37° C.

### C) Secretion Trap Assay

The secretion trap was performed as follows: Media was aspirated from the wells and then the cells were fixed for 15 minutes with 1.8% formaldehyde in PBS. Cells were then washed with TNT (0.1M Tris-HCL, 0.15M NaCl, and 0.05% Tween-20 in H₂O), and permeabilized with 0.1% Triton-X in PBS for 15 minutes, and again washed with TNT. Cells were blocked for 1 hour with TNB (0.1M Tris-HCL, 0.15M NaCl and 0.5% Blocking Reagent (NEN Renaissance TSA-Direct Kit) in H₂O), and washed again with TNT. The cells were incubated for 1 hour with 2 μg/ml mzB7R1/Vasp-biotin soluble receptor fusion protein. Cells were then washed with TNT. Cells were fixed a second time for 15 minutes with 1.8% formaldehyde in PBS. After washing with TNT, cells were incubated for another hour with 1:1000 diluted streptavidin HRP. Again cells were washed with TNT.

Positive binding was detected with fluorescein tyramide reagent diluted 1:50 in dilution buffer (NEN kit) and incubated for 5 minutes, and washed with TNT. Cells were preserved with Vectashield Mounting Media (Vector Labs Burlingame, Calif.) diluted 1:5 in TNT. Cells were visualized using a FITC filter on fluorescent microscope.

### Example 7

### zB7R1-VASP in Acute Graft Versus Host Disease (GVHD)

The purpose of this experiment was to determine if prophylactic treatment of B7R1-VASP soluble protein influences the development and severity of an acute GVHD response in mice.

To initiate GVHD, 75 million spleen cells from C57B1/6 mice are injected by intravenous delivery into DBA2 X C57B1/6 F1 mice (BDF1) on day 0. Mice are treated with 150 ug of B7R1-VASP protein intraperitoneally every other day starting the day before cell transfer and continuing throughout the duration of the experiment. Body weight is monitored daily and mice are sacrificed on day 12 after spleen transfer. Spleens are collected for FACS analysis and blood is collected for serum. Prophylactic delivery of B7R1-VASP significantly decreases the severity of body weight loss during acute GVHD.

### Example 8

### B7R1 is Regulated in Tissues from Mice with Collagen Induced Arthritis (CIA) Compared to Non-Disease Tissue

Experimental Protocol: Tissues were obtained from mice with varying degrees of disease in the collagen-induced arthritis (CIA) model. The model was performed following standard procedures of immunizing male DBA/1J mice with collagen (see Example 9 below) and included appropriate non-diseased controls. Tissues isolated included affected paws and popliteal lymph nodes. RNA was isolated from all tissues using standard procedures. In brief, tissues were collected and immediately frozen in liquid N2 and then transferred to −80° C. until processing. For processing, tissues were placed in Qiazol reagent (Qiagen, Valencia, Calif.) and RNA was isolated using the Qiagen Rneasy kit according to manufacturer's recommendations. Expression of murine zB7R1 mRNA was measured with multiplex real-time quantitative RT-PCR methods (TaqMan) and the ABI PRISM 7900 sequence detection system (PE Applied Biosystems). Murine zB7R1 mRNA levels were normalized to the expression of murine hypoxanthine guanine physphoribosyl transferase mRNA and determined by the comparative threshold cycle method (User Bullein 2: PE Applied Biosystems).

Results: Murine B7R1 mRNA expression was detected in the tissues tested. Higher levels of expression were observed in lymph nodes compared to the paws. B7R1 mRNA was increased in the popliteal lymph nodes and the paws from mice in the CIA model of arthritis compared to tissues obtained from non-diseased controls, and the levels were associated with disease severity. B7R1 mRNA was increased in the paws approximately 2.3-fold in mice with mild disease and approximately 4-fold in mice with severe disease compared to non-diseased controls. B7R1 mRNA was increased in the lymph node approximately 1.5-fold in mice with mild disease and approximately 1.8-fold in mice with severe disease compared to non-diseased controls.

### Example 9

### B7R1m-mFc and B7R1m-VASP CH6 Decreases Disease Incidence and Progression in Mouse Collagen Induced Arthritis (CIA) Model

Mouse Collagen Induced Arthritis (CIA) Model: Ten week old male DBA/1J mice (Jackson Labs) were divided into 3 groups of 13 mice/group. On day-21, animals were given an intradermal tail injection of 50-100 μl of 1 mg/ml chick Type II collagen formulated in Complete Freund's Adjuvant (prepared by Chondrex, Redmond, Wash.), and three weeks later on Day 0 they were given the same injection except prepared in Incomplete Freund's Adjuvant. B7R1m-mFc or B7R1m-VASP CH6 was administered as an intraperitoneal injection every other day for 1.5 weeks (although dosing may be extended to as must as four weeks), at different time points ranging from Day-1 to a day in which the majority of mice exhibit moderate symptoms of disease. Groups received 150 μg of B7R1m-mFc or B7R1m-VASP CH6 per animal per dose, and control groups received the vehicle control, PBS (Life Technologies, Rockville, Md.). Animals began to show symptoms of arthritis following the second collagen injection, with most animals developing inflammation within 1.5-3 weeks. The extent of disease was evaluated in each paw by using a caliper to measure paw thickness, and by assigning a clinical score (0-3) to each paw: 0=Normal, 0.5=Toe(s) inflamed, 1=Mild paw inflammation, 2=Moderate paw inflammation, and 3=Severe paw inflammation as detailed below.

Monitoring Disease: Animals can begin to show signs of paw inflammation soon after the second collagen injection, and some animals may even begin to have signs of toe inflammation prior to the second collagen injection. Most animals develop arthritis within 1-3 weeks of the boost injection, but some may require a longer period of time. Incidence of disease in this model is typically 95-100%, and 0-2 non-responders (determined after 6 weeks of observation) are typically seen in a study using 40 animals. Note that as inflammation begins, a common transient occurrence of variable low-grade paw or toe inflammation can occur. For this reason, an animal is not considered to have established disease until marked, persistent paw swelling has developed.

All animals were observed daily to assess the status of the disease in their paws, which is done by assigning a qualitative clinical score to each of the paws. Every day, each animal had its 4 paws scored according to its state of clinical disease. To determine the clinical score, the paw can be thought of as having 3 zones, the toes, the paw itself (manus or pes), and the wrist or ankle joint. The extent and severity of the inflammation relative to these zones was noted including: observation of each toe for swelling; torn nails or redness of toes; notation of any evidence of edema or redness in any of the paws; notation of any loss of fine anatomic demarcation of tendons or bones; evaluation of the wrist or ankle for any edema or redness; and notation if the inflammation extends proximally up the leg. A paw score of 1, 2, or 3 is based first on the overall impression of severity, and second on how many zones are involved. The scale used for clinical scoring is shown below.

Clinical Score:
0=Normal
0.5=One or more toes involved, but only the toes are inflamed
1=mild inflammation involving the paw (1 zone), and may include a toe or toes
2=moderate inflammation in the paw and may include some of the toes and/or the wrist/ankle (2 zones)
3=severe inflammation in the paw, wrist/ankle, and some or all of the toes (3 zones)

Established disease is defined as a qualitative score of paw inflammation ranking 2 or more, that persists for two days in a row. Once established disease is present, the date is recorded and designated as that animal's first day with “established disease”.

Blood is collected throughout the experiment to monitor serum levels of anti-collagen antibodies, as well as serum immunoglobulin and cytokine levels. Serum anti-collagen antibodies correlate well with severity of disease Animals are euthanized on a determined day, and blood collected for serum. From each animal, one affected paw may be?? collected in 10% NBF for histology and one is frozen in liquid nitrogen and stored at −80° C. for mRNA analysis. Also, ½ spleen, ½ thymus, ½ mesenteric lymph node, one liver lobe and the left kidney are collected in RNAlater for RNA analysis, and ½ spleen, ½ thymus, ½ mesenteric lymph node, the remaining liver, and the right kidney are collected in 10% NBF for histology. Serum is collected and frozen at −80° C. for immunoglobulin and cytokine assays.

Groups of mice that received soluble zB7R1-Fc fusion protein as described herein and zB7R1-VASP CH6 as described herein, at all time points tested (prophylactic and therapeutic delivery) were characterized by a delay in the incidence (for prophylactic administration), onset and/or progression of paw inflammation. On day 8 of the model, mice that received PBS prophylactically had 100% disease incidence and had significant swelling of the majority of their paws. However, mice that received zB7R1-Fc fusion protein prophylactically had significantly reduced paw swelling (2.3-fold lower arthritis score compared to PBS-treated mice) and 80% incidence. Moreover, mice treated prophlyactically with zB7R1-VASP CH6 fusion protein were greatly protected from disease, as only 40% of these mice developed arthritis symptoms, which was associated with markedly reduced arthritis scores (3.5-fold lower than PBS-treated mice). zB7R1-VASP CH6 fusion protein was also able to reduce arthritis symptoms when administered after disease onset, such that mice treated therapeutically with zB7R1-VASP CH6 fusion protein had approximately 2-fold lower arthritis scores than mice treated therapeutically with PBS. These results indicate that soluble zB7R1 fusion proteins of the present invention reduce inflammation, as well as disease incidence and progression.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

We claim:

1. A method of preparing a tetrameric protein comprising:
culturing a host cell transformed or transfected with an expression vector encoding a fusion protein comprising a vasodialator-stimulated phosphoprotein (VASP) domain and a heterologous protein.

2. The method of claim 1, wherein the heterologous protein comprises an extracellular domain of a transmembrane protein.

3. The method of claim 1, wherein the fusion protein is soluble.

4. The method of claim 1, wherein the VASP domain is derived from the human VASP gene.

5. The method of claim 4, wherein the VASP domain comprises amino acids 5 to 38 of SEQ ID NO:2.

6. The method of claim 1, wherein the fusion protein further comprises a linker sequence.

7. A fusion protein produced by the method of claim 1.

8. A fusion protein comprising a VASP domain and a heterologous protein.

9. The protein of claim 8, wherein the heterologous protein comprises the extracellular domain of a B7 family member.

10. The protein of claim 9, wherein the B7 family member is B7H1 or zB7R1.


SEQUENCE LISTING (19 sequences)

SEQ ID NO: 1 - DNA, 147 bp, Artificial Sequence
  VASP-His6 tetramerizing domain
ggctccggtggctccgacctacagagggtgaaacaggagcttctggaagaggtgaagaaggaattgcagaaagtgaaagaggaaatcattgaagccttcgtccaggagctgaggggttccggtggccatcaccatcaccatcactga

SEQ ID NO: 2 - protein, 48 aa, Artificial Sequence
  VASP-His6 tetramerizing domain
GSGGSDLQRVKQELLEEVKKELQKVKEEIIEAFVQELRGSGGHHHHHH

SEQ ID NO: 3 - DNA, 103 bp, Artificial Sequence
  primer
acgcttccgtagatctggttccggaggctccggtggctccgacctacagagggtgaaacaggagcttctggaagaggtgaagaaggaattgcagaagtgaaag

SEQ ID NO: 4 - DNA, 103 bp, Artificial Sequence
  primer
aaggcgcgcctctagatcagtgatggtgatggtgatggccaccggaacccctcagctcctggacgaaggcttcaatgatttcctctttcactttctgcaattc

SEQ ID NO: 5 - DNA, 47 bp, Artificial Sequence
  primer
ctcagccaggaaatccatgccgagttgagacgcttccgtagatctgg

SEQ ID NO: 6 - DNA, 47 bp, Artificial Sequence
  primer
ggggtggggtacaaccccagagctgttttaaggcgcgcctctagatc

SEQ ID NO: 7 - DNA, 44 bp, Artificial Sequence
  primer
ccacaggtgtccagggaattcgcaagatgaggatatttgctgtc

SEQ ID NO: 8 - DNA, 34 bp, Artificial Sequence
  primer
ctccggaaccagatctttcatttggaggatgtgc

SEQ ID NO: 9 - DNA, 873 bp, Artificial Sequence
  B7H1-VASP-His6
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaagatctggttccggaggctccggtggctccgacctacagagggtgaaacaggagcttctggaagaggtgaagaaggaattgcagaaagtgaaagaggaaatcattgaagccttcgtccaggagctgaggggttccggtggccatcaccatcaccatcactga

SEQ ID NO: 10 - protein, 290 aa, Artificial Sequence
  B7H1-VASP-His6
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERSGSGGSGGSDLQRVKQELLEEVKKELQKVKEEIIEAFVQELRGSGGHHHHHH

SEQ ID NO: 11 - protein, 4 aa, Artificial Sequence
  linker
GSGG

SEQ ID NO: 12 - protein, 6 aa, Artificial Sequence
  tag
HHHHHH

SEQ ID NO: 13 - DNA, 873 bp, Homo sapiens
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaa

SEQ ID NO: 14 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 15 - protein, 8 aa, Artificial Sequence
  tag
DYKDDDDK

SEQ ID NO: 16 - DNA, 2207 bp, Homo sapiens
ccccttcctgtggggttcattggggcatcccctttctgctgcaggaacctctcatcagaccgcctgagggaagcggcgcccggagacccgccccggcccggtccacattctccccaggaagccggactctatggggcgggaccctgggggagcctgagccgagcccggagccagccccgaacccctgaacctccagccaggggcgccccgggagcagccagcccgtgggcgagccgcccgcccgccgagcagccatgagcgagacggtcatctgttccagccgggccactgtgatgctttatgatgatggcaacaagcgatggctccctgctggcacgggtccccaggccttcagccgcgtccagatctaccacaaccccacggccaattcctttcgcgtcgtgggccggaagatgcagcccgaccagcaggtggtcatcaactgtgccatcgtccggggtgtcaagtataaccaggccacccccaacttccatcagtggcgcgacgctcgccaggtctggggcctcaacttcggcagcaaggaggatgcggcccagtttgccgccggcatggccagtgccctagaggcgttggaaggaggtgggccccctccacccccagcacttcccacctggtcggtcccgaacggcccctccccggaggaggtggagcagcagaaaaggcagcagcccggcccgtcggagcacatagagcgccgggtctccaatgcaggaggcccacctgctccccccgctgggggtccacccccaccaccaggacctccccctcctccaggtccccccccacccccaggtttgcccccttcgggggtcccagctgcagcgcacggagcagggggaggaccaccccctgcaccccctctcccggcagcacagggccctggtggtgggggagctggggccccaggcctggccgcagctattgctggagccaaactcaggaaagtcagcaagcaggaggaggcctcaggggggcccacagcccccaaagctgagagtggtcgaagcggaggtgggggactcatggaagagatgaacgccatgctggcccggagaaggaaagccacgcaagttggggagaaaacccccaaggatgaatctgccaatcaggaggagccagaggccagagtcccggcccagagtgaatctgtgcggagaccctgggagaagaacagcacaaccttgccaaggatgaagtcgtcttcttcggtgaccacttccgagacccaaccctgcacgcccagctccagtgattactcggacctacagagggtgaaacaggagcttctggaagaggtgaagaaggaattgcagaaagtgaaagaggaaatcattgaagccttcgtccaggagctgaggaagcggggttctccctgaccacagggacccagaagacccgcttctcctttccgcacacccggcctgtcaccctgctttccctgcctctacttgacttggaattggctgaagacacaggaatgcatcgttcccactccccatcccacttggaaaactccaagggggtgtggcttccctgctcacacccacactggctgctgattggctggggaggcccccgcccttttctccctttggtccttcccctctgccatccccttggggccggtccctctgctggggatgcaccaatgaaccccacaggaagggggaaggaaggagggaatttcacattcccttgttctagattcactttaacgcttaatgccttcaaagttttggtttttttaagaaaaaaaaatatatatatatttgggttttgggggaaaagggaaatttttttttctctttggttttgataaaatgggatgtgggagtttttaaatgctatagccctgggcttgccccatttggggcagctatttaaggggaggggatgtctcaccgggctgggggtgagatatccccccaccccagggactccccttccctctggctccttccccttttctatgaggaaataagatgctgtaactttttggaacctcagttttttgattttttatttgggtaggttttggggtccaggccattttttttaccccttggaggaaataagatgagggagaaaggagaaggggaggaaacttctcccctcccaccttcacctttagcttcttgaaaatgggcccctgcagaataaatctgccagtttttataaaaaaaaaa

SEQ ID NO: 17 - protein, 380 aa, Homo sapiens
MSETVICSSRATVMLYDDGNKRWLPAGTGPQAFSRVQIYHNPTANSFRVVGRKMQPDQQVVINCAIVRGVKYNQATPNFHQWRDARQVWGLNFGSKEDAAQFAAGMASALEALEGGGPPPPPALPTWSVPNGPSPEEVEQQKRQQPGPSEHIERRVSNAGGPPAPPAGGPPPPPGPPPPPGPPPPPGLPPSGVPAAAHGAGGGPPPAPPLPAAQGPGGGGAGAPGLAAAIAGAKLRKVSKQEEASGGPTAPKAESGRSGGGGLMEEMNAMLARRRKATQVGEKTPKDESANQEEPEARVPAQSESVRRPWEKNSTTLPRMKSSSSVTTSETQPCTPSSSDYSDLQRVKQELLEEVKKELQKVKEEIIEAFVQELRKRGSP

SEQ ID NO: 18 - protein, 244 aa, homo sapians
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGAYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 19 - protein, 140 aa, homo sapians
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGAYTGRIFLEVLESSVAEHGARFQI